(19) United States
(12) Patent Application Publication
(10) Pub. No.: US 2026/0034485 A1
(43) Pub. Date: Feb. 5, 2026

(54) DISPOSABLE FILTER CAPSULE ASSEMBLY

(71) Applicant: Saint-Gobain Life Sciences (Hangzhou) Co., Ltd., Hangzhou (CN)

(72) Inventors: Liqing HONG, Hangzhou (CN); Yong ZHANG, Hangzhou (CN)

(21) Appl. No.: 19/109,899

(22) PCT Filed: Oct. 31, 2023
(86) PCT No.: PCT/CN2023/128363

§ 371 Date: Mar. 7, 2025

(30) Foreign Application Priority Data
Sep. 9, 2022 (CN) 202211103760.6

(51) Int. Cl. B01D 35/30; A61M 16/00; A61M 16/08; A61M 16/10; B01D 29/13; B01D 39/16

(52) U.S. Cl. CPC B01D 35/30; A61M 16/0003; A61M 16/0808; A61M 16/085; A61M 16/105; B01D 29/13; B01D 39/1607; B01D 2257/80

(57) ABSTRACT

A disposable filter capsule assembly to secure a tube to the outer surface of the assembly. The filter capsule assembly includes a single or multiple-layer filter membrane secured in the assembly. Tube clips are formed on a surface of the filter capsule assembly to secure an attached tube. The tube clips can be formed with clip openings in stacked and tandem arrangements to secure multiple coils or multiple tubes, wires or similar elongate components.

### CROSS-REFERENCE TO RELATED APPLICATIONS

None.

### FIELD OF THE DISCLOSURE

The disclosure relates to filter capsule apparatus with external tube, pipe and wire securing means. More particularly, the disclosure concerns disposable filter capsules with surface mounted tube and wire retaining components to secure tubes and other system components in a compact, protected assembly.

### BACKGROUND OF THE DISCLOSURE

Patients requiring breathing assistance may be assisted by one of a plethora of breathing assistance devices such as nebulizers, oxygen concentrators and ventilators. When supplemental oxygen or other gases and/or medications is/are administered to patients, whether it be via nasal canula (high or low flow), Venturi mask or intubation (ventilator), the supplemental oxygen, oxygen-enhanced air or modified gas combinations are often infused with various medicines and therapeutic gases such as Nitric Oxide. To ensure the proper therapeutic gases and medicines are administered in therapeutic doses, sensors are used to detect the various contents of the gas mixture in a breathing circuit to ensure proper administration. To this end, sampling lines are installed in breathing apparatus to direct samples from the breathing circuit to the sensors. Sensors often can be negatively impacted by humidity or the presence of water in the samples derived from the breathing circuit. Filters and related devices are used to remove the water component from the samples to protect the samples from damage and/or malfunction. The ability of the sensors to accurately identify the presence and concentration of medicines and therapeutic gases is critical.

One subset of patients particularly in need of accurate aerosolized medicine and therapeutic gases are newborns and infants with compromised pulmonary function. One condition known as hypoxic respiratory failure concerns infants that present with difficulty breathing on their own. This leads to reduced oxygen levels in tissues throughout the body. This can lead to pulmonary hypertension, respiratory distress syndrome and pneumonia among other unfortunate conditions.

One therapy used to counter the problems associated with hypoxic respiratory failure is to introduce nitric oxide in a gaseous form into an infant's respiratory system. Nitric oxide is well known to increase blood oxygen levels by inducing vasodilation when administered to a patient in a therapeutic dose. By increasing blood oxygen levels, oxygen infusion into the various tissues is improved. For infants, this is accomplished with a ventilator that functions as a bellows to move air or gaseous mixtures in an out of the patient's respiratory system. The volume delivered as well as other parameters of the air or air/gas mixture can be precisely controlled with a ventilator.

To introduce nitric oxide gas into an infant, the gas is mixed with oxygen and delivered via a ventilator. One of the parameters that has to be controlled with the delivery of the NO/O₂ mixture with respect to sampling is the humidification level of the gaseous mixture derived from the breathing circuit. This is particularly important with infants, and especially so with intubated infants, due to the delicate ciliary structures of the newly formed lungs and the related pleural tissues. There are at least two methods used conventionally to address humidification levels in samples taken from a breathing circuit.

The first method is to use a filter apparatus to filter out aerosolized water droplets and water condensed from a vapor form in a gas sample with the use of hydrophobic filter media. Although effective to remove some of the water component from a gas, hydrophobic filters cannot remove gas-phase water. Moreover, filter apparatuses with hydrophobic filters are not effective to precisely control the amount of moisture or gas-phase water resident in a gas.

The second known method to control humidification in a gas is to employ a Nafion™ tube. Nafion™ tubes are formed from a copolymer of tetrafluoroethylene (Teflon®) and another fluorocarbon chain having sulfonic acid groups secured to side chains. Among other properties imparted by the presence of the sulfonic acid groups, the fluorocarbon polymer readily absorbs water whether in a vapor or in a liquid phase. Although most of the fluorocarbon polymer is hydrophobic in nature, the sulfonic acid groups form ionic channels through the polymer that permits the transport of water through the polymer. In this manner, the polymer functions like a hydrophobic semi-permeable membrane that can selectively trap water.

Unlike filter membranes that trap molecules based upon the size of the molecules, Nafion™ tubes transfer water molecules from one side of the polymer material to the other via kinetic reaction. By transferring and removing water vapor via chemical reactivity from gaseous materials, analytes in the gas stream are essentially unaffected by the tubes. The driving force that causes the Nafion™ tubes to transfer water vapor is the partial pressure of water vapor on either side of the tube/membrane. The tubes transfer water vapor until the partial pressure of water vapor on either side of the tube/membrane reaches equilibrium. This provides the advantageous ability to selectively remove water vapor without significantly retaining or retarding the transfer of various gases in the mixture, e.g., Nitrogen (N₂) and Oxygen (O₂), and/or Oxides, e.g., Carbon Dioxide (CO₂) and Nitric Oxide (NO).

Although both methods of humidification control provide effective means to control water vapor content in sampled gas mixtures, each method has its shortcomings and drawbacks. Although hydrophobic filters are quite effective to remove condensed water and aerosolized water droplets from gases, the amount of water captured is difficult to control with a membrane-based filter. Membrane-based filters will capture essentially any water in a gas to produce a “dry” gas. For gases to be inhaled by a patient and monitored by a physician, this is problematic because gas samples taken from a breathing circuit need to be free of the presence of water/humidity to ensure the sensors can function properly and accurately.

With respect to Nafion™ tubes, there are structural limits to how much humidity, i.e., liquid-phase water, aerosolized water droplets and water vapor, can be removed (or added) to a gas mixture. The tubes are able to alter the humidification level of a gas by only about plus or minus 10 to 20 percent. The larger the tube, the more water vapor can be transported into or out of a gas passing through the tube. How large a tube can be used is limited by the dimensional limitations of the apparatus to which the Nafion™ tubes are attached, i.e., the ventilator assemblies.

To solve the limitations of both the filter capsule and Nafion™ tube humidification control methods, it has been found that the combination of a gas permeable filter capsule with a Nafion™ tube provides superior humidity removal and control. How the two components are connected is a problem yet to be solved. What is needed is a filter capsule apparatus that can connect to and contain a Nafion™ tube in a compact enclosure so as to protect the tube and any associated assemblies from damage or malfunction due to loose connection or arrangement of the tube relative to the filter capsule. What also is needed is a means to secure a Nafion™ tube so as to maintain the tube's exposure to environmental conditions to enable the humidification control function of the tube. These and other objects of the disclosure will become apparent from a reading of the following summary and detailed description of the disclosure as well as a review of the appended drawings.

### SUMMARY OF THE DISCLOSURE

The filter capsule assemblies disclosed herein comprise different embodiments, each of the embodiments including a filter housing or shell containing a filter element therein that may be planar, toroidal, pleated, singular or multilayer or formed in any of the filter configurations known in the art. All of the embodiments have an inlet port extending into the upstream or inlet side of the shell, and an outlet port extending into the downstream or outlet side of the shell. The terms “inlet,” “inlet side,” “upstream,” “upstream side,” and similar terms all refer to the portion or volume of the filter assembly located on the inlet portion of the device, i.e., between the filter housing or shell and the outer surface of the filter element containing unfiltered liquid during operation of the filter. The terms “outlet,” “outlet side,” “downstream,” “downstream side,” and similar terms all refer to the portion or volume of the filter assembly located on the outlet portion of the apparatus, between the filter housing or shell and the outer surface of the filter element containing filtered liquid that has passed through the filter element during operation of the filter capsule assembly. The filter element defines the gas permeable or liquid permeable barrier between the upstream or inlet side and the downstream or outlet side of the assembly. Thus, all fluids introduced into the filter capsule assembly must pass through the filter element from the inlet port to the outlet port of the filter capsule assembly.

In one aspect of the disclosure, a combined filter capsule and Nafion™ tube includes a filter capsule assembly having a filter capsule inlet shell half formed with a barbed inlet port extending from an inlet end. The filter capsule assembly further includes a filter capsule outlet shell half having features for registration against the filter capsule inlet shell half. The filter capsule shell halves, when registered together, form a filter chamber. A dual membrane filter is secured in the filter chamber between the filter capsule inlet shell half and the filter capsule outlet shell half. An outlet port is formed on the filter capsule outlet shell half with features to secure a Nafion™ tube both mechanically and functionally.

In another aspect of the disclosure, the filter capsule outlet shell half is formed with at least one tube-retaining clip to secure a Nafion™ tube to the filter capsule assembly. The tube-retaining clip may be formed from materials that exhibit rigid properties, e.g., polypropylene, or may be formed from thermoplastic materials with pliability characteristics that enable flexion of the clip to facilitate insertion of one or more tubes and rebound to maintain registration with, and retention of, the Nafion™ tube that changes in cross-sectional diameter with the absorption of water in vapor or liquid form. This permits the Nafion™ tube to expand and retract without being restricted from expanding or disengaging from the filter capsule assembly with shrinkage. These and other aspects of the disclosure will become apparent from a review of the appended drawings and a reading of the following detailed description of the disclosure.

### BRIEF DESCRIPTION OF THE DRAWINGS

FIG. 1 is a side view in elevation of a filter capsule assembly with a modified outlet port and tube-receiving clips according to one embodiment of the disclosure.

FIG. 2 is a side view in elevation of the filter capsule assembly shown in FIG. 1 with the view rotated 90° relative to the FIG. 1 view.

FIG. 3 is a top, side perspective view of the filter capsule assembly shown in FIG. 1.

FIG. 4 is a side sectional view in elevation of the filter capsule assembly shown in FIG. 1.

FIG. 5 is a side view in elevation of a filter capsule outlet shell half according to the embodiment of the disclosure shown in FIG. 1.

FIG. 6 is a top, side perspective view of the filter capsule outlet shell half shown in FIG. 5.

FIG. 7 is a side sectional view in elevation of an outlet shell half/tube clip subassembly according to a further embodiment of the disclosure.

FIG. 8 is a top, side perspective view of the filter capsule assembly shown in FIG. 1 with a Nafion™ tube secured with tube clips.

FIG. 9 is a top, side perspective view of the filter capsule assembly shown in FIG. 1 with a multi-coiled Nafion™ tube secured with tube clips.

### DETAILED DESCRIPTION OF THE DISCLOSURE

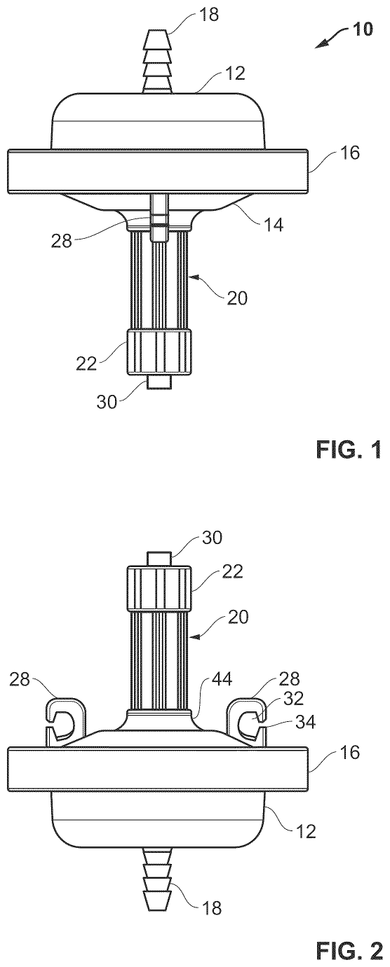

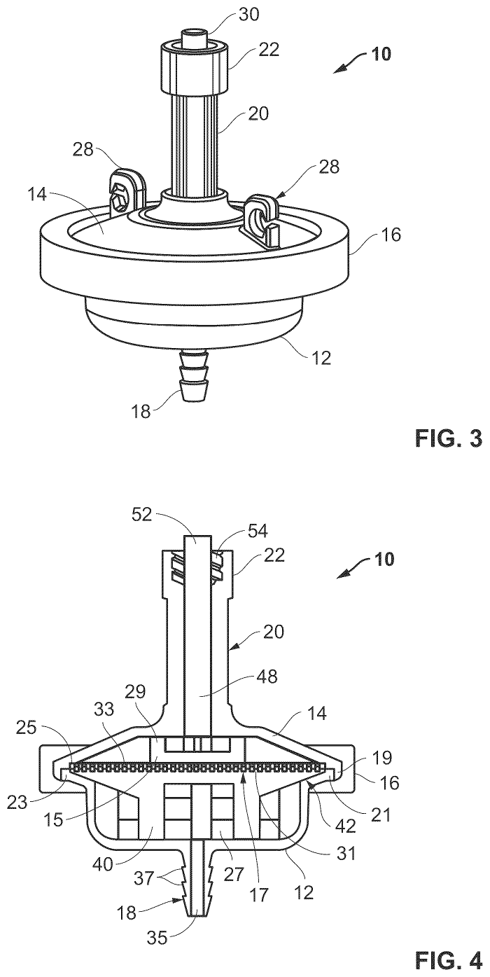

Referring to FIGS. 1-6, in one aspect of the disclosure, a filter capsule assembly is shown designated generally as 10. Filter capsule assembly 10 includes a filter capsule inlet shell half 12 formed in the shape of a cylindrical cup that defines a first part of a filter chamber. Any shape may be used for the inlet shell half and remain within the scope of the disclosure. The depth of inlet shell half 12 is dimensioned to hold a volume of gas, gas mixture and/or gas/liquid mixture so that the liquid component of any mixture can be collected away from the enclosed filter membrane surface. The depth of inlet shell half 12 can be varied to accommodate different fluid volumes depending upon the application. Any fluid introduced into filter capsule assembly 10 may be subject to variable residency time so as to accommodate different flow rates.

Filter capsule assembly 10 further includes a filter capsule outlet shell half 14 formed with a tapered annular wall that transitions to an outlet port 20 disclosed in more detail hereinbelow. Outlet shell half 14 defines a second part of a filter chamber. Assembly of inlet shell half 12 to outlet shell half 14 defines filter chamber 15 dimensioned to receive and secure a filter element designated generally as 17 (as shown in FIG. 4) and disclosed in more detail hereinbelow. It should be understood that outlet shell half 14 does not have to be tapered and may be formed with a variety of cross-sectional shapes that further define filter chamber 15.

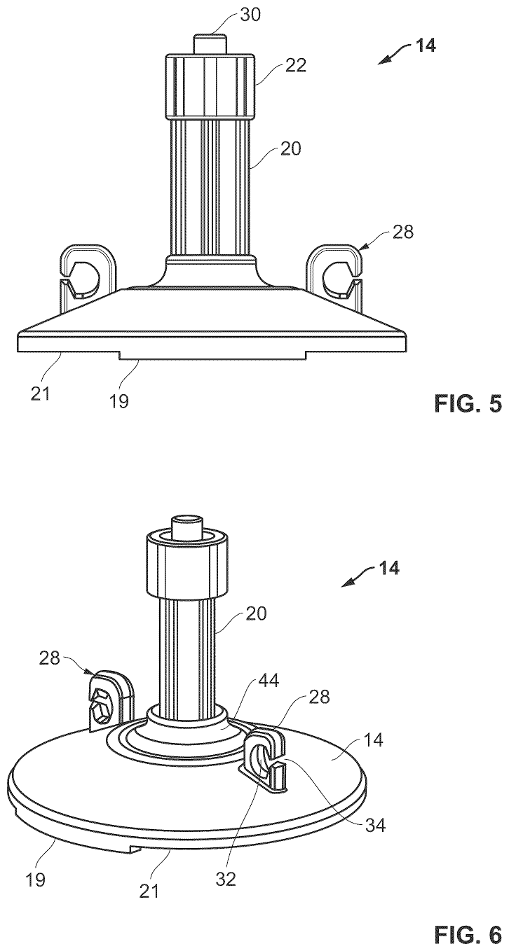

The registration surfaces of inlet shell half 12 and outlet shell half 14 may be planar or may include interlocking shell wall extensions to mechanically orient the rotational orientation of the shell halves to each other. As shown in FIGS. 5 and 6, outlet shell half 14 includes two diametrically opposed outlet rim extensions 19 that correspond to the annular contour of the shell half and extend axially from an edge of the shell half. Outlet rim extensions 19 further define outlet rim channels 21 that extend between outlet rim extensions 19. Inlet shell half 12 also may be formed with pairs of inlet rim extensions and inlet rim channels dimensioned to correspond to, and interlock with, outlet rim extensions 19 and outlet rim channels 21. To perform the interlocking function, each outlet rim extension 19 corresponds to, and registers with, a dedicated inlet rim channel and each outlet rim channel 21 corresponds to, and registers with, a dedicated inlet rim extension. In this manner the orientation of the corresponding rim extensions and rim channels creates a mechanical lock between the shell halves.

To permanently secure the shell halves together, a rim band 16 is formed about the perimeter edges of the assembled filter capsule shell halves. Rim band 16 may be formed by overmolding the assembled shell halves' perimeter edges with polymeric material to lock the shell halves in registration. It should be understood that the shell halves may be joined by other means such as by, thermal welding, spin welding, laser welding, adhesive or sonic welding and remain within the scope of the disclosure.

Inlet shell half 12 includes an annular filter element inlet support shelf 23 formed about the perimeter of the shell half. If inlet shell half 12 is formed with inlet rim extensions and inlet rim channels, inlet support shelf 23 is set radially inwardly from the inlet rim extensions and channels. Outlet shell half 14 is formed with an annular filter element outlet support shelf 25 formed about the perimeter of the shell half. If outlet shell half 14 is formed with outlet rim extensions 19 and outlet rim channels 21, outlet support shelf 25 is set radially inwardly from the outlet rim extensions and channels. When the shell halves are assembled, inlet support shelf 23 and outlet support shelf 25 support, secure and suspend a filter media element 17 within filter chamber 15 so as to form upstream and downstream sub-chambers defined by filter media element 17. An upstream sub-chamber 27 is defined by the inner wall surface of inlet shell half 12 and the upstream side of filter media element 17. A downstream sub-chamber 29 is defined by the inner wall surface of outlet shell half 14 and the downstream side of filter media element 17.

Filter media element 17 may be formed illustratively as a single layer, disc-shaped filter or be formed from multiple layers, each identical to the other layers with respect to material or each formed from different materials to impart different filtering characteristics. It should be understood that alternative types of filter elements may be used with respect to shapes and constructions, e.g., woven, nonwoven, stacked disc, toroidal-shaped filters, pleated filters including pleated filter cartridges, tubular, hollow fibers and even contained loose filter media and remain within the scope of the disclosure. Filter media element 17 also may be constructed from a variety of materials including polymeric, ceramic and metallic. In one illustrative, nonlimiting embodiment, a dual membrane has a first filter membrane layer 31 formed from glass fiber and a second filter membrane layer 33 formed from Polytetrafluoroethylene (PTFE).

As shown in FIG. 4, the first filter membrane layer 31 is shown upstream of second filter membrane layer 33. It should be understood that the orientation of the filter membrane layers may be reversed with the second filter membrane layer upstream the first filter membrane layer and remain within the scope of the disclosure. It should be understood further that any porosity and pore size(s) may be selected for any layer of filter membrane 17 to be the same or different relative to the other layers (if any) and remain within the scope of the disclosure.

When the intended purpose of filter capsule assembly 10 is to filter out liquids, such as water in liquid phase and aerosolized water droplets, the materials selected to form filter membrane 17 are intended to be hydrophobic in function to block the passage of any aerosolized water droplets or water in liquid phase in a gas or gas mixture flowed through filter capsule assembly 10. It should be understood that the materials selected can impart other characteristics such as oleophobicity and remain within the scope of the disclosure.

Illustrative filtration materials that exhibit hydrophobic properties suitable for the filter capsule assembly disclosed herein include Polytetrafluoroethylene (PTFE), Polyvinylidene Fluoride (PVDF), glass fiber and polyethylene. It should be understood this list is nonlimiting. Any filter material exhibiting hydrophobic properties may be used to form filter media element 17. Moreover, it should be further understood that hydrophilic materials may be selected to form filter media element 17 should an application require the passage of liquid and the retention of gaseous materials including gas in the form of gas bubbles. In addition, hybrid materials exhibiting both hydrophobic and hydrophilic properties may be used, any combination of which remains within the scope of the disclosure.

More particularly, filter media element 17 may be constructed from fibrous material, including, but not limited to, microfibers and nanofibers of polyethylene, polypropylene, nylon, polyester, carbon, fiberglass, polypropylene sulfide (PPS), Polytetrafluoro-ethylene (Teflon® PTFE), polyvinylidene fluoride (PVDF), polyacrylonitrile (PAN), Ethylene chlorotrifluoroethylene (ECTFE), polyethylene/ultra-high molecular weight polyethylene (PE/UPE) including cellulose/diatomaceous earth or silica blends, cellulose/carbon particles or fibers, cellulose/ion exchange resins, cellulose acetate, nitrocellulose as are available from general media suppliers, as well as combinations of any of the disclosed filter media materials.

Still further filter materials may include microporous, hydrophilic or hydrophobic membranes, including, but not limited to, materials such as polyethersulfone, polysulfone, cellulose acetate, polyvinylidene fluoride (PVDF), and other fluoropolymers such as perfluoroalkoxy (PFA) and its derivatives, MFA (co-polymer of tetrafluoroethylene and perfluoromethyl vinyl ether and sold under the name Hyflon), fluorinated ethylene propylene polymer (FEP) and the like, as well as combinations of any of the disclosed filter media materials.

Filter media element 17 may be constructed from a number of manufacturing processes including, but not limited to, wet-laid processes (similar to papermaking), wet casting, melt-cast, or dry processes such as air-laid, melt-blown, spun-bond, bi-directional starching, etc. as is well known in the art.

Referring still to FIGS. 1-6, inlet shell half 12 has an inlet port 18 that extends axially from the shell half. Inlet port 18 defines an inlet port channel 35 in fluid communication with upstream sub-chamber 27. An outer surface of inlet port 18 may be formed with one or more barbs 37 to secure tubing (not shown) that delivers fluid materials, e.g., gas mixtures of O₂ and NO, into filter capsule assembly 10. It should be understood that the surface of inlet port 18 may be smooth and remain within the scope of the disclosure. Inlet port 18 may include other tube connecting means such as luer lock structures and quick connects (not shown) as are well known in the art.

Inlet shell half 12 may be formed with a plurality of reinforcing posts 40 positioned about the inner perimeter of the shell half to rigidify the shell half. Tapered post extensions 42 may extend radially outwardly toward inlet support shelf 23 to provide further structural support. In an alternative embodiment, inlet shell half 12 may be formed without any structural supports and be formed with smooth inner wall surfaces that terminate in a membrane-supporting annular shelf.

Extending axially from outlet shell half 14 is outlet port 20. A base of outlet port 20 may be reinforced with port collar 44. An outlet port distal end 30 of the outlet port may be formed with luer lock connector 22 having internal luer lock threading 54 to permit filter capsule apparatus 10 to be secured to a larger assembly such as a ventilator or to a Nafion™ tube among two illustrative, nonlimiting examples. It should be understood that other connection means may be used for outlet port 20 including illustratively, quick connects and barbed fittings, and remain within the scope of the disclosure. Outlet port 20 defines an outlet channel 48 that extends the length of the outlet port and may be open at outlet port distal end 30. Outlet channel 38 in fluid communication with downstream sub-chamber 29 and in fluid communication with any components secured to outlet port distal end 30.

By attaching a Nafion™ tube to outlet port 20 via luer lock 22 (or other connection means), any gases filtered by filter capsule assembly 10 can be further processed by being transferred through the tube before being transferred to a larger assembly such as a ventilator or sensor apparatus. In this manner, the humidity level of the gases/gas mixtures filtered by filter capsule assembly 10 can be more completely and precisely controlled by adding the humidification controlling function of the Nafion™ tube. Depending upon the humidity level of the tube's environment, the humidity level of the filtered gases/gas mixtures can be increased or decreased through the tube's function to equilibrate the humidity level between its environment and any fluids (gas and/or liquid) transferred through the tube. This advantageously permits humidity level control without compromising any analytes in the pre-filtered gas/gas mixture entering the Nafion™ tube.

To secure the body of a Nafion™ tube apart from the ends secured to ports including outlet port 20 (connection not shown), one or more tube clips 28 are formed on a surface of outlet shell half 14. Tube clip(s) 28 extend(s) axially from the shell half and are positioned radially inwardly from the perimeter edge of outlet shell half 14. Each tube clip 28 defines a clip opening 32 that may be substantially circular in shape. A tube insertion slot 34 creates a discontinuity in the tube clip portions that define clip opening 32. Insertion slot 34 permits either distortion of the tube clip or provides a tube entrance point to permit the body of a Nafion™ tube to be inserted into the clip until clip opening 32 is occupied by the tube. It should be understood that the tube clips can be formed on any part of the shell including the inlet shell half and remain within the scope of the disclosure.

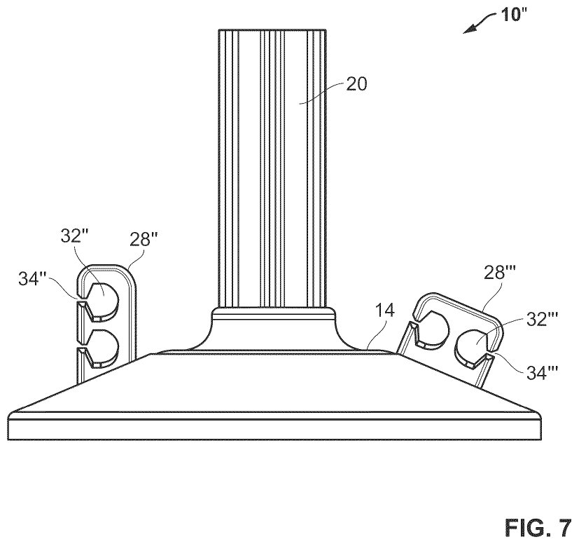

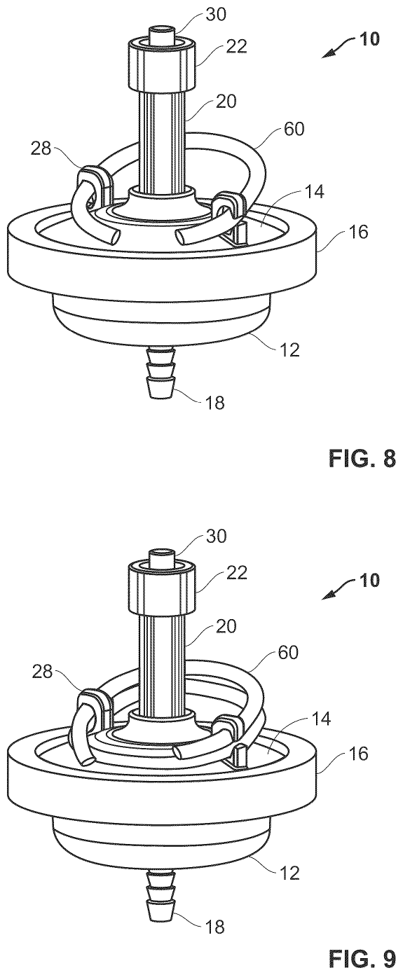

Referring now to FIGS. 7-9, each tube clip may be formed with multiple clip openings and clip slots as shown in FIG. 7 to permit the coiled stacking of a Nafion™ tube 60 (or other elongate component) to permit different lengths of Nafion™ tubes or multiple tubes to be attached to, and coiled about, filter capsule assembly 10. In an alternative embodiment, each clip opening can be dimensioned to receive multiple coils or loops of tubing 60 or similar elongate material such as wiring as shown in FIG. 9. As is well known in the art, the amount of gas-phase water a Nafion™ tube can absorb is directly proportion to the dimensions (width and length) of the tube. By providing stacked tube clips 28″ with axially stacked and spaced clip openings 32″ and corresponding insertion slots 34″, filter capsule assembly 10 can accommodate differing lengths of Nafion™ tubes to impart different magnitudes of humidity control. As used herein, any feature designated by a primed reference character corresponds, structurally or functionally, to a feature of a different filter assembly embodiment designated by the same reference character, unprimed or differently primed. It should be understood that tandem tube clips (designated as 28′″ in FIG. 7) also can be formed with tandem or horizontally arranged clip openings 32″ with corresponding insertion slots 34′″ formed on opposing sides of the tube clip to provide additional and alternate tube-mounting means.

The materials used to construct the filter cups, capsules, shell halves and other non-filter element components may be the same for all these components. The components may be injection molded with any thermal plastic materials, including, but not limited to, Polypropylene (PP), Polyethylene (PE), Nylon, Polysulfone, Perfluoroalkoxy (PFA) polymer resin, Polycarbonate (PC), PS, Polyethersulfone (PES), Ethylene-chlorotrifluoroethylene copolymer (ECTFE) and mixtures thereof. It should be understood other materials and manufacturing methods well known in the art also may be used to construct these components.

While the present disclosure has been described in connection with several embodiments thereof, it will be apparent to those skilled in the art that many changes and modifications may be made without departing from the true spirit and scope of the present disclosure. Accordingly, it is intended by the appended claims to cover all such changes and modifications as come within the true spirit and scope of the disclosure.

What I claim as new and desire to secure by United States Letters Patent is:

1. A disposable filter capsule assembly comprising:
a two-piece shell comprising an inlet shell half and an outlet shell half secured together, wherein the inlet shell half and the outlet shell half define a filter chamber;
a filter media element secured in the filter chamber, wherein an upstream surface of the filter media element and an inner surface of the inlet shell half define an upstream sub-chamber, and wherein a downstream surface of the filter media element and an inner surface of the outlet shell half define a downstream sub-chamber;
an inlet port extending from the top shell half, wherein the inlet port is in fluid communication with the upstream sub-chamber;
an outlet port extending from the bottom shell half, wherein the outlet port is in fluid communication with the downstream sub-chamber; and,
at least one tube clip formed on an outer surface of the lower shell half.

2. The filter capsule assembly of claim 1 wherein the at least one tube clip defines a clip opening and an insertion slot.

3. The filter capsule assembly of claim 2 further comprising a tube, wherein the tube is secured in the clip opening.

4. The filter capsule assembly of claim 3 wherein the filter media element has a structure selected from the group consisting of single layer membrane, dual-layer membrane, stacked disc, planar, toroidal, pleated membrane, pleated filter cartridge, tubular, hollow fiber, contained loose filter media and combinations thereof.

5. The filter capsule assembly of claim 4 wherein the filter media element is formed from a material selected from the group consisting of fibrous material including microfibers and nanofibers of polyethylene, polypropylene, nylon, polyester, carbon, fiberglass, polypropylene sulfide (PPS), Polytetrafluoro-ethylene (Teflon® PTFE), polyvinylidene fluoride (PVDF), polyacrylonitrile (PAN), Ethylene chlorotrifluoroethylene (ECTFE), polyethylene/ultra-high molecular weight polyethylene (PE/UPE) including cellulose/diatomaceous earth and silica blends, cellulose/carbon particles and fibers, cellulose/ion exchange resins, cellulose acetate and nitrocellulose, and microporous, hydrophilic and hydrophobic membranes formed from materials including polyethersulfone, polysulfone, cellulose acetate, polyvinylidene fluoride (PVDF), perfluoroalkoxy (PFA) and its derivatives, MFA (co-polymer of tetrafluoroethylene and perfluoromethyl), vinyl ether, fluorinated ethylene propylene polymer (FEP) and combinations thereof.

6. The filter capsule assembly of claim 1 wherein the filter media element has a structure selected from the group consisting of single layer membrane, dual-layer membrane, stacked disc, planar, toroidal, pleated membrane, pleated filter cartridge, tubular, hollow fiber, contained loose filter media and combinations thereof.

7. The filter capsule assembly of claim 1 comprising a plurality of tube clips extending axially from an outer surface of the outlet shell half and positioned radially inwardly from a perimeter edge of the outlet shell half.

8. A disposable filter capsule assembly comprising:
a two-piece shell comprising an inlet shell half and an outlet shell half secured together, wherein the inlet shell half and the outlet shell half define a filter chamber;
a filter media element secured in the filter chamber, wherein an upstream surface of the filter media element and an inner surface of the inlet shell half define an upstream sub-chamber, and wherein a downstream surface of the filter medial element and an inner surface of the outlet shell half define a downstream sub-chamber;
an inlet port extending from the top shell half, wherein the inlet port is in fluid communication with the upstream sub-chamber;
an outlet port extending from the bottom shell half, wherein the outlet port is in fluid communication with the downstream sub-chamber; and,
at least one tube clip formed on an outer surface of the lower shell half, wherein the clip comprises at least two clip openings and two insertion slots each connected to a clip opening.

9. The filter capsule assembly of claim 8 wherein the clip openings are stacked or vertically-oriented clip openings.

10. The filter capsule assembly of claim 8 wherein the clip openings are tandem or horizontally-oriented clip openings.

11. The filter capsule assembly of claim 8 further comprising a tube, wherein the tube is secured in at least one of the two clip openings.

12. The filter capsule assembly of claim 8 comprising a plurality of tube clips extending axially from an outer surface of the outlet shell half and positioned radially inwardly from a perimeter edge of the outlet shell half.

13. The filter capsule assembly of claim 12 further comprising at least one tube secured in the plurality of tube clips.

14. The filter capsule assembly of claim 13 wherein the filter media element has a structure selected from the group consisting of single layer membrane, dual-layer membrane, stacked disc, planar, toroidal, pleated membrane, pleated filter cartridge, tubular, hollow fiber, contained loose filter media and combinations thereof.

15. The filter capsule assembly of claim 14 wherein the filter media element is formed from a material selected from the group consisting of fibrous material including microfibers and nanofibers of polyethylene, polypropylene, nylon, polyester, carbon, fiberglass, polypropylene sulfide (PPS), Polytetrafluoro-ethylene (Teflon® PTFE), polyvinylidene fluoride (PVDF), polyacrylonitrile (PAN), Ethylene chlorotrifluoroethylene (ECTFE), polyethylene/ultra-high molecular weight polyethylene (PE/UPE) including cellulose/diatomaceous earth and silica blends, cellulose/carbon particles and fibers, cellulose/ion exchange resins, cellulose acetate and nitrocellulose, and microporous, hydrophilic and hydrophobic membranes formed from materials including polyethersulfone, polysulfone, cellulose acetate, polyvinylidene fluoride (PVDF), perfluoroalkoxy (PFA) and its derivatives, MFA (co-polymer of tetrafluoroethylene and perfluoromethyl), vinyl ether, fluorinated ethylene propylene polymer (FEP) and combinations thereof.

16. (canceled)

17. (canceled)

18. The filter capsule assembly of claim 13 further comprising a plurality of tubes secured in the plurality of tube clips.

19. The filter capsule assembly of claim 8 further comprising a single tube secured to the at least one tube clip, wherein the single tube is coiled to form a multi-coiled tube with multiple coils, and wherein the multiple coils are secured to the at least one tube clip.

20. The filter capsule assembly of claim 8 further comprising a single tube secured to the at least one tube clip, wherein the single tube is coiled to form a multi-coiled tube with multiple coils, and wherein each coil of the multiple coils is secured in a dedicated clip opening.
